(19) United States
(12) Patent Application Publication
(10) Pub. No.: US 2026/0188427 A1
(43) Pub. Date: Jul. 2, 2026

(54) PRECISE DE NOVO SEQUENCING METHOD FOR TOP-DOWN PROTEOMICS

(71) Applicant: The Florida State University Research Foundation, Inc., Tallahassee, FL (US)

(72) Inventor: Lissa Caitlin Anderson, Tallahassee, FL (US)

(21) Appl. No.: 19/545,945

(22) Filed: Feb. 20, 2026

Related U.S. Application Data
(63) Continuation of application No. PCT/US2025/043557, filed on Aug. 26, 2025.
(60) Provisional application No. 63/687,068, filed on Aug. 26, 2024.

(51) Int. Cl. G16B 30/00; G06F 7/483; G16B 40/10

(52) U.S. Cl. CPC G16B 30/00; G06F 7/4833; G16B 40/10

(57) ABSTRACT

Computerized methods and systems of de novo sequencing from a mass spectrometer and identifying a biological polymer using mass invariant charge patterns in the spectrometer data by transforming spectra to a natural logarithmic space where peaks arising from the same analyte mass align along a predictable pattern defined solely by charge state. In some embodiments, the computerized method employs an operation that iterates the residue mass in the transformed natural logarithmic space, e.g., minimizing charge state difference errors between corresponding isotopologues assigned to different charge states. In some embodiments, the de novo sequencing of the present disclosure also allows for viewing the mass-to-charge (m/z) spectrum in a natural logarithmic manner (e.g., Equation 1 −ln(m/z−q)) to provide confidence in any reassignment of peaks in an observed charge pattern vector.

### RELATED APPLICATION

This is a continuation application of PCT Application No. PCT/US2025/043557, filed Aug. 26, 2025, entitled, “A PRECISE DE NOVO SEQUENCING METHOD FOR TOP-DOWN PROTEOMICS,” which claims priority to, and the benefit of, U.S. Provisional Patent Application No. 63/687,068, filed Aug. 26, 2024, entitled “PRECISE DE NOVO SEQUENCING METHOD FOR TOP-DOWN PROTEOMICS,” each of which is incorporated by reference herein in its entirety.

### STATEMENT REGARDING FEDERALLY SPONSORED RESEARCH

This invention was made with Government Support under Grant No. DMR-2128556 awarded by the National Science Foundation (NSF). The Government has certain right in the invention.

### FIELD

The present disclosure relates to methods of de novo sequencing of peptides and proteins from isotopically resolved mass spectra without the need for m/z-to-mass deconvolution via averagine fitting.

### BACKGROUND

Regulation of nearly every cellular process is directly linked to the primary structure of the proteins involved. Comprehensive knowledge of protein primary structure cannot be derived from the genome because translation of mRNA into protein may not dictate the chemical composition of the final protein product. The ultimate mass spectrometry (MS)-based proteomics platform would be capable of unequivocally distinguishing closely related protein sequences while concurrently characterizing any post-translational modifications (PTMs), which requires intact protein analysis (top-down proteomics, TDP).

Intact protein analysis presents persistent challenges due to the complexity of isotope distributions arising from the incorporation of heavy isotopes of carbon, hydrogen, nitrogen, oxygen, and sulfur (FIG. 1). As protein mass increases, the probability of multiple heavy isotope incorporations rises, diminishing the relative abundance of the monoisotopic peak. In many cases-especially for low-abundance species or spectra with poor signal-to-noise—the monoisotopic peak may be weak or entirely undetectable. This complicates the process of determining accurate molecular mass, which is foundational to all top-down proteomic workflows.

A common workaround is to estimate the monoisotopic mass using the “averagine” model, which approximates an average elemental composition for amino acids to predict expected isotope distributions (Senko et al. 1995). While convenient, averagine-based fits can be shifted by one or more isotopologues if the monoisotopic peak is absent or misidentified, introducing systematic mass errors of 1-2 Da or more (FIG. 2). To accommodate these errors, database search tolerances must be widened, increasing the risk of false positives. Conversely, narrowing mass tolerances improves specificity but excludes true matches when mass estimates are inaccurate due to incorrect isotopic modeling. Additionally, mass measurement accuracy is inherently limited by the discrepancy between the true elemental composition of the analyte and the assumptions built into the averagine model. This issue is further compounded by electrospray ionization (ESI), which generates multiple charge states for each analyte, splitting signal intensity across overlapping mass-to-charge ratio (m/z) values, requiring high-resolution and spectral averaging for accurate deconvolution.

Current protein identification strategies—whether based on database search, spectral libraries, or de novo sequencing—depend heavily on the accuracy of these monoisotopic mass assignments (Nesvizhkii 2010). In database-driven approaches, spectra are m/z-to-mass deconvolved via averagine fits, and the resulting experimental monoisotopic masses are compared against in silico predictions from protein sequence databases. This inherently limits identification to sequences already present in the database. As a result, proteoforms containing unexpected sequence variants or post-translational modifications (PTMs) are not accurately identified or remain unidentified (Smith). For samples derived from non-model organisms or for applications requiring discovery of novel proteoforms—such as antibody sequencing—de novo approaches are the only viable option.

However, top-down de novo sequencing itself remains limited by the same assumptions: most methods still rely on averagine-based deconvolution to derive fragment and precursor monoisotopic masses prior to sequence inference. The inability to reliably determine monoisotopic mass without prior compositional knowledge hinders both identification and accurate interpretation of spectra. Thus, there is a need for systems and methods thereof to improve top-down proteomic sequencing analyses.

The systems and methods of the present disclosure address these needs.

### SUMMARY

The present disclosure provides computerized methods of de novo sequencing from a mass spectrometer and identifying a biological polymer using mass invariant charge patterns in the spectrometer data by transforming spectra to a natural logarithmic space where peaks arising from the same analyte mass align along a predictable pattern defined solely by charge state. In some embodiments, the computerized method employs an operation that iterates residue mass in the transformed natural logarithmic space for sequence inference. In some embodiments, the de novo sequencing of the present disclosure also allows for viewing the mass-to-charge (m/z) spectrum in a natural logarithmic manner (e.g., Equation 1-ln(m/z−q)) to provide confidence in any reassignment of peaks in an observed charge pattern vector.

The present disclosure also provides computerized methods of internally calibrating or correcting raw spectra from a mass spectrometer, wherein the calibration or correction of the raw spectra is based on the mass invariant charge pattern.

The present disclosure provides systems and non-transitory computer-readable medium (CRM) for performing de novo sequencing.

In one aspect, disclosed herein is a computerized method of de novo sequencing of a biological polymer from a mass spectrometer, the method comprising:
obtaining (or providing) a data file comprising a raw mass-to-charge (m/z) spectrum and inputting the data file into a processor;
performing a peak-picking algorithm by the processor to extract one or more centroided peaks from the raw mass-to-charge (m/z) spectrum;
performing a transformation operation of a m/z value of the one or more centroided peaks into a charge-dependent value that enables invariant comparison between the one or more centroided peaks;
generating a charge pattern vector by the processor, wherein the one or more peaks are grouped to match a spacing pattern across multiple charge states, and wherein one or more integer charge states are assigned to the one or more peaks based on their position within a matched pattern;
generating from the processor nearby fragment peak clusters relative to a control peak with a defined threshold; and
assembling fragment peak clusters, via the processor, from consecutive residue mass shifts to identify a full or partial sequence of a biological polymer by a de novo sequencing operation.

In some embodiments, the raw mass-to-charge (m/z) spectrum or spectra of the mass spectrometer is provided internally, e.g., as a data object, e.g., wherein the mass spectrometer equipment is configured to perform the exemplary method. Data object may be an internal file or an internal data used natively in the equipment.

In some embodiments, the transformation operation comprises applying a natural logarithmic function in the form of Equation 1:

$$ln ( \frac{m}{z} - q ) , \qquad Equation 1$$

wherein q is a charge carrier mass.

In some embodiments, the biological polymer is sequenced by matching residue mass differences between consecutive fragment peak clusters. In some embodiments, the biological polymer comprises a sequence of unknown composition. In some embodiments, the biological polymer comprises a polypeptide (including, but not limited to an antibody, a glycoprotein, a hormone, an enzyme, a contractile protein, a structural protein, a storage protein, or a fragment thereof), a polynucleotide (including, but not limited to deoxyribonucleic acid (DNA), ribonucleic acid (RNA), a chemically modified analog, or a fragment thereof), or a fragment thereof.

In some embodiments, the method further comprises identifying one or more post-translation modifications of the biological polymer or the unknown biological polymer. In some embodiments, the method further comprises identifying one or more amino acid or nucleotide substitutions. In some embodiments, the method further comprises identifying one or more isoforms of the biological polymer.

In some embodiments, the charge carrier comprises an electron, a proton, a monoatomic ion, or a polyatomic ion. In some embodiments, the raw profile-mode spectrum is generated from a tandem (MS/MS) mass spectrometer. In some embodiments, the method directly identifies an amino acid sequence tag directly from charge-resolved isotopologue peaks, without requiring monoisotopic mass assignment, collapsing isotopologue clusters into a deconvolved mass spectrum, or database matching. In some embodiments, the method directly identifies a polynucleotide sequence tag directly from charge-resolved isotopologue peaks, without requiring monoisotopic mass assignment, collapsing isotopologue clusters into a deconvolved mass spectrum, or database matching.

In some embodiments, the method comprises internally calibrating the spectrum by minimizing charge state difference errors between corresponding isotopologues assigned to different charge states. In some embodiments, the method identifies and removes a false peak that does not conform to predicted charge state or mass difference patterns from the raw profile-mode spectrum. In some embodiments, the method is used for drug testing, drug discovery, contaminant detection, clinical diagnostics, identification of pathological molecules, biomarkers, or a combination thereof. In some embodiments, the method is coupled to an additional analytical method comprising gas chromatography, liquid chromatography, spectroscopy, microscopy, or a combination thereof.

In some aspects, disclosed herein is a method of identifying a biological polymer, the method comprising performing a de novo sequencing method comprising the steps of any preceding aspect.

In some aspects, disclosed herein is a computerized method of internally calibrating or correcting a raw mass-to-charge (m/z) spectrum, the method comprising:
obtaining (or providing) a data file comprising a raw mass-to-charge (m/z) spectrum and entering the data file into a processor;
performing a peak-picking algorithm by the processor to extract one or more centroided peaks from the raw mass-to-charge (m/z) spectrum;
performing a transformation operation of a m/z value of the one or more centroided peaks into a charge-dependent value that enables invariant comparison between the one or more centroided peaks;
generating a charge pattern vector by the processor, wherein the one or more peaks are grouped to match a log-space spacing pattern across multiple charge states, and wherein one or more integer charge states are assigned to the one or more peaks based on their position within a matched pattern;
calculating expected positions of peaks based on a calibration model comprising a frequency-to-m/z conversion equation; and
adjusting one or more parameters of the calibration model to minimize deviations between observed and expected charge patterns, thereby internally calibrating the raw spectrum without the use of external calibrants.

In some embodiments, the method is used prior to sequencing of a biological polymer. In some embodiments, the raw m/z spectrum is generated from the biological polymer. In some embodiments, the biological polymer comprises more than one charge. In some embodiments, the biological polymer comprises a polypeptide (including but not limited to an antibody, a glycoprotein, a hormone, an enzyme, a contractile protein, a structural protein, a storage protein, or a fragment thereof), a polynucleotide (including but not limited to deoxyribonucleic acid (DNA), ribonucleic acid (RNA), a chemically modified analog, or a fragment thereof), or a fragment thereof.

In some embodiments, the raw m/z spectrum is generated from a tandem (MS/MS) mass spectrometer. In some embodiments, the method improves mass accuracy and improves sequence alignments generated after calibration. In some embodiments, the method minimizes deviations in ln-space charge spacing between corresponding isotopologues assigned to different charge states. In some embodiments, the method detects and removes a false peak that does not conform to predicted charge spacing or frequency-m/z relationships. In some embodiments, the method is used for drug testing, drug discovery, contaminant detection, clinical diagnostics, identification of pathological molecules, or a combination thereof.

In some embodiments, a calibration logic is implemented in software having computer-executable instructions configured to preprocess mass spectra from an ion-trapping instrument. In some embodiments, calibration is applied during acquisition in real time.

In some aspects, disclosed herein is a system comprising:
at least one processor; and
a memory operably coupled to the at least one processor, wherein the memory has computer executable instructions stored thereon that, when executed by at least one processor, cause at least one processor to:
receive a data file comprising a raw mass-to-charge (m/z) spectrum
apply a peak-picking algorithm to extract one or more centroided peaks from the raw m/z spectrum;
perform a transformation operation of a mass-to-charge (m/z) value into a charge-dependent value that enables invariant comparison between the one or more centroided peaks;
generate a charge pattern vector, wherein the one or more peaks are grouped to match a log-space spacing pattern across multiple charge states, and wherein one or more integer charge states are assigned to the one or more peaks based on their position within a matched pattern;
output nearby fragment peak clusters relative to a control peak with a defined threshold; and
assemble fragment peak clusters from consecutive residue mass shifts to identify a biological polymer.

In some aspects, disclosed herein is a non-transitory computer-readable medium (CRM) having instructions stored thereon, wherein execution of the instructions by a processor causes the processor to:
receive a data file comprising a raw mass-to-charge (m/z) spectrum
apply a peak-picking algorithm to extract one or more centroided peaks from the raw m/z spectrum;
perform a transformation operation of a mass-to-charge (m/z) value into a charge-dependent value that enables invariant comparison between the one or more centroided peaks;
generate a charge pattern vector, wherein the one or more peaks are grouped to match a log-space spacing pattern across multiple charge states, and wherein one or more integer charge states are assigned to the one or more peaks based on their position within a matched pattern;
output nearby fragment peak clusters relative to a control peak with a defined threshold; and
assemble fragment peak clusters from consecutive residue mass shifts to identify a biological polymer.

In some embodiments, the system and/or the non-transitory CRM comprises a transformation operation that comprises a function in the form of Equation 1:

$$ln ( \frac{m}{z} - q ) , \qquad Equation 1$$

wherein q is a charge carrier mass.

### BRIEF DESCRIPTION OF FIGURES

The accompanying figures, which are incorporated in and constitute a part of this specification, illustrate several aspects described below.

FIG. 1 (top left) shows the isotope distribution of the 50+ charge state of protein AG (50.4 kDa), and (top right) the isotope distribution of the 10+ charge state of ubiquitin (8.6 kDa). Monoisotopic m/z's are indicated along with, for some signals, the number of additional neutrons (Neu) present due to incorporation of heavy isotopes of C, H, N, O, and S. The bottom of FIG. 1 shows the monoisotopic mass and mass measurement accuracies for both proteins based on elemental composition (EC) and averagine fit by the Xtract algorithm (Thermo Fisher Scientific).

FIG. 2 shows the mass measurement accuracy of fragment ions after m/z-to-mass deconvolution using a published charge-pattern-based algorithm (FLASHDeconv). Data are derived from a 1500-scan averaged ETD MS/MS spectrum of bovine Carbonic Anhydrase II (29 kDa) acquired following 6 ms electron-transfer dissociation. Fragment ions were processed by decharging and deisotoping under standard algorithm parameters. The scatter plot illustrates mass error distribution for matched c- and z•-type fragments, color-coded by tolerance: ±10 ppm (gray), ±1.1 Da (blue), and ±2.2 Da (purple). Signals inconsistent with expected fragment patterns are indicated in red (adapted from Weisbrod et al. 2017, and FLASHDeconv algorithm as described by Jeong et al. 2020).”

FIGS. 3A, 3B, 3C, 3D, and 3E show the use of mass-invariant charge pattern in ln(m/z−q) space for charge state assignment and precision assessment. FIG. 3A shows the charge state distribution of Protein G (21.4 kDa) taken by 21-tesla FT-ICR MS as the sum of four transients. FIG. 3B shows the expanded view of the 16+ charge state with the three most abundant isotopologues indicated with asterisks. FIG. 3C shows the natural log-transformed values of the three most abundant isotopologues for charge states 13+ to 26+. FIG. 3D shows that the selected ln(m/z−q) values are given and match theoretical values to the sixth decimal place. FIG. 3E shows the natural log-transformed mass difference measurement accuracy expressed in ppm.

FIGS. 4A and 4B show the internal calibration of an FT-ICR MS/MS spectrum using the mass-invariant charge pattern in ln(m/z−q) space. FIG. 4A shows the mass measurement accuracy versus m/z for the five most abundant isotopologues of charge states 12+ to 22+ of intact Apomyoglobin (spectrum not shown) before (red) and after charge-state-difference calibration (blue). FIG. 1B shows that iterating the B-term in the Ledford equation to minimize charge state difference error (purple) reflects improved consistency in the expected invariant charge pattern, while the concurrent dip in mass error demonstrates enhanced mass measurement accuracy (green).

FIG. 5 shows the sequence tag inference from an FT-ICR MS/MS spectrum of Protein G using the charge-resolved, natural log-transformed mass-difference framework. (Left) The table shows m/z and ln(m/z−q) values for the three most abundant isotopologues of seven consecutive fragments from the ion series (charge state 11+). Predicted ln(m/z−q)ₙ₋₁ values were calculated from the observed (m/z)ₙ using Equation 7 and compared to observed values to assess isotopologue correspondence. The observed ln(m/z−q)ₙ₋₁ values (yellow) are matched to the predicted values (green). (Right) Expanded views of the corresponding fragment ion series from a 21 T FT-ICR CID MS/MS spectrum of Protein G (post-FT average of 3 scans, 150,000 resolving power at m/z 400). A ten-residue sequence tag (VETVMETVTF (SEQ ID NO: 1)) was derived from the 11+ ions and independently confirmed using the 12+ series.

FIG. 6 shows the discrimination of near-isobaric residues lysine (K) and glutamine (Q) in a single-scan CID MS/MS spectrum of Carbonic Anhydrase II (29 kDa). (Top) The table shows m/z and ln(m/z−q) values for the five most abundant consecutive isotopologues of three consecutive fragments (charge state 20⁺). Predicted ln(m/z−q)ₙ₊₁ values were calculated from the observed (m/z)ₙ using Equation 7 and compared to observed values to assess isotopologue correspondence. The observed ln(m/z−q)ₙ₊₁ values (yellow) are matched to the predicted values (green). (Bottom) Mass-scale expanded view of a 20⁺ fragment ion series from a single-transient CID MS/MS spectrum of Carbonic Anhydrase II acquired on a 21 T FT-ICR. Despite imperfect isotope distribution fidelity and interfering peaks, the precision with which the experimental data matches the predicted ln(m/z−q) values enables distinction of Q versus K residues.

FIGS. 7A and 7B show a ln-transformed charge-mass plot of the 34⁺ precursor of Carbonic Anhydrase II (29 kDa) following ETD (Weisbrod et. al. 2017). FIG. 7A shows the expanded view of the boxed region in FIG. 7B. Dashed lines connect fragments observed in multiple charge states. Fragment masses were calculated for charge-assigned fragments with Equation 8 and plotted against their ln(m/z−q) values, revealing distinct curves that correspond to the charge states of the fragment ion series. “X” symbols represent theoretical monoisotopic peaks of manually identified fragments. Circles indicate charge assigned isotopologues matched to the sequence using ExD Viewer (Beckman et al. 2021).o

FIG. 8 shows the expanded view of individual isotopologues from the natural log-transformed charge-mass plot of Carbonic Anhydrase II (29 kDa) following ETD (Weisbrod et. al.). This figure shows a zoomed in region of the shaded area from FIG. 7A, highlighting the isotopologue distributions of selected fragment ions. An additional c-ion that was not manually identified is revealed (O only), albeit with fewer than expected isotopologues, suggesting low signal-to-noise. A z•-ion peak cluster that was not correctly charge-assigned by ExDViewer (x only) was also observed.

FIG. 9A shows sequence tag inference from an FT-ICR MS/MS spectrum of Thioredoxin using the charge-resolved, natural log-transformed mass-difference framework. The top shows the expanded view of the fragment ion series from a 21 T FT-ICR CID MS/MS spectrum of Thioredoxin (single transient acquisition, 150,000 resolving power at m/z 400). The table (bottom) shows m/z and ln(m/z−q) values for the four most abundant isotopologues of ten consecutive fragments from the ion series (charge state 7⁺). Predicted ln(m/z−q)ₙ₊₁ values were calculated from the observed (m/z)ₙ using Equation 7 and compared to observed values to assess isotopologue correspondence. The observed ln(m/z−q)ₙ₊₁ values (yellow) match the predicted values (green) for a minimum of two out of the four isotopologues. A nine-residue sequence tag (AXEYEVSAV (SEQ ID NO: 14)) was derived.

FIG. 9B shows a table of the data of FIG. 9A.

FIG. 10A shows a table of simulated peptide charge states (given along the top), and m/z values. FIG. 10B shows fit of the mass invariant charge pattern vector to the simulated peptides in panel A. It is evident that, regardless of the mass or identity of the peptide or protein, the spacing between consecutive charge states remains the same in the natural-log transformed space.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances, and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof, as used herein, are used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

As used in this disclosure and in the appended claims, the singular forms “a,” “an,” “the,” include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Comprising” is intended to mean that the compositions, methods, etc., include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate-buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

As used herein, “proteomics” or “proteomic sequencing” refers to a field of study focused on detecting and identifying the amino acid sequence of proteins, and further allows one of skill in the art to characterize said proteins, including their primary amino acid sequence, potential secondary and tertiary structures, post-translational modifications, and other protein variations (such as isoforms).

“De novo sequencing” refers to a method used to determine the initial sequencing of a nucleic acid or amino acid sequence when a reference sequence is not available or known. The said method involves assembling overlapping DNA or peptide fragments to construct a contiguous and/or complete sequence.

Reference is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, typically of a length of 20 or fewer amino acids, and more typically of a length of 12 or fewer amino acids (Garrett & Grisham, Biochemistry, 2nd edition, 1999, Brooks/Cole, 110).

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

Reference is also made herein to nucleotides, nucleic acids, polynucleotides, and compositions comprising nucleotides, nucleic acids, and polynucleotides. As used herein, a “nucleotide” refers to a compound consisting of a nucleoside, which consists of a nitrogenous base and a 5-carbon sugar, linked to a phosphate group, forming the basic structural unit of nucleic acids, such as DNA or RNA. The five types of nucleotides are adenine (A), cytosine (C), guanine (G), thymine (T), and uracil (U), each of which is bound together by a phosphodiester bond to form a nucleic acid molecule. Thus, a “polynucleotide” refers to a polymer composed of nucleotide monomers that are covalently bonded to form a chain. Typically, polynucleotides range from a few nucleotides (5-10 in length) to a whole chromosome comprising billions of nucleotides. As used herein, the terms “polynucleotide” and “oligonucleotide” can be used interchangeably, wherein an oligonucleotide typically contains about 2 nucleotides to 100 nucleotides.

A “nucleic acid” is a chemical compound that serves as the primary information-carrying molecule in cells and makes up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

### Computerized Methods of De Novo Sequencing and Systems Thereof

The present disclosure provides computerized methods of de novo sequencing from a mass spectrometer and identifying a biological polymer using the computerized method of de novo sequencing. The present disclosure also provides computerized methods of internally calibrating or correcting raw spectra from a mass spectrometer.

In one aspect, disclosed herein is a computerized method of de novo sequencing of a biological polymer from a mass spectrometer, the method comprising:
obtaining (or providing) a data file or data object comprising a raw mass-to-charge (m/z) spectrum;
performing a peak-picking algorithm by the processor to extract one or more centroided peaks from the raw mass-to-charge (m/z) spectrum;
performing a transformation operation of a m/z value of the one or more centroided peaks into a charge-dependent value that enables invariant comparison between the one or more centroided peaks;
generating a charge pattern vector by the processor, wherein the one or more peaks are grouped to match a spacing pattern across multiple charge states, and wherein one or more integer charge states are assigned to the one or more peaks based on their position within a matched pattern;
generating from the processor nearby fragment peak clusters relative to a control peak with a defined threshold; and
assembling fragment peak clusters, via the processor, from consecutive residue mass shifts to identify a full or partial sequence of a biological polymer by a de novo sequencing operation.

In some embodiments, the transformation operation comprises applying a natural logarithmic function in the form of Equation 1 or its equivalence:

$$ln ( \frac{m}{z} - q ) , \qquad Equation 1$$

wherein q is a charge carrier mass.

In some embodiments, the biological polymer is sequenced by matching residue mass differences between consecutive fragment peak clusters. In some embodiments, the biological polymer comprises a sequence of unknown composition. As used herein, a “biological polymer” or “biopolymer” refers to large macromolecules comprising smaller repeating units referred to as “monomers”. Said biological polymers of the present disclosure include, but are not limited to, polynucleotides, polypeptides, polysaccharides, and lipids, which are made up of nucleotides, amino acids, sugars, and fatty acids, respectively. Thus, in some embodiments, the computerized method, system, and/or CRM of any aspect disclosed herein comprises performing de novo sequencing on a biological polymer, including, but not limited to, polynucleotides, polypeptides, polysaccharides, and lipids. In some embodiments, the biological polymer comprises a polypeptide (including, but not limited to an antibody, a glycoprotein, a hormone, an enzyme, a contractile protein, a structural protein, a storage protein, or a fragment thereof), a polynucleotide (including, but not limited to deoxyribonucleic acid (DNA), ribonucleic acid (RNA), a chemically modified analog, or a fragment thereof), or a fragment thereof.

In some embodiments, the method further comprises identifying one or more post-translation modifications (including, but not limited to phosphorylation, acylation(s), alkylation(s), glycosylation, ubiquitination, oxidation, biotinylation, and nitrosylation) of the biological polymer or the unknown biological polymer. In some embodiments, the method further comprises identifying one or more amino acid or nucleotide mutations, such as, for example, substitutions, additions/insertions, deletions, and indels (insertion-deletions). As used herein, a “mutation” refers to changing the structure of a gene, resulting in a variant form that may be transmitted to later generations. A mutation is caused by the alteration of single nucleotides in DNA, or the deletion, insertion, or rearrangement of larger sections of genes. A mutation can lead to the expression of a protein that has been changed physically or functionally, leading to lethality, non-lethal dysfunction effects, or no effects. Thus, the present disclosure provides computerized methods and systems thereof for identifying nucleotide and/or amino acid mutations.

In some embodiments, the method further comprises identifying one or more isoforms of the biological polymer. As used herein, an “isoform” refers to direct forms of a polypeptide or polynucleotide that are derived from the same gene but have slightly different amino acid sequences or nucleotide sequences. Said isoforms may be derived from alternative splicing of the primary RNA transcript or through the use of differing transcription start sites or differing termination sites. Thus, the present disclosure provides computerized methods and systems thereof to identify gene or protein products of alternative splicing.

In some embodiments, the charge carrier comprises an electron (a negative charge carrier), a proton (a positive charge carrier), a monoatomic ion (a charge carrier comprising one atom), or a polyatomic ion (a charge carrier comprising two or more atoms). In some embodiments, the raw profile-mode spectrum is generated from a tandem (MS/MS) mass spectrometer. The present disclosure provides that the computerized method of any aspect disclosed herein can be performed using any mass spectrometer known in the art. In some embodiments, the method directly identifies an amino acid sequence tag from charge-resolved isotopologue peaks, without requiring monoisotopic mass assignment, collapsing isotopologue clusters into a deconvolved mass spectrum, or database matching. In some embodiments, the method directly identifies a polynucleotide sequence tag directly from charge-resolved isotopologue peaks, without requiring monoisotopic mass assignment, collapsing isotopologue clusters into a deconvolved mass spectrum, or database matching.

In some embodiments, the method comprises internally calibrating the spectrum by minimizing charge state difference errors between corresponding isotopologues assigned to different charge states. In some embodiments, the method identifies and removes a false peak that does not conform to predicted charge state or mass difference patterns from the raw profile-mode spectrum, a centroided spectrum, or peak list (in the event that the method occurs after peak picking). In some embodiments, the method is used for drug testing, drug discovery, contaminant detection, clinical diagnostics, identification of pathological molecules, biomarkers, or a combination thereof. In some embodiments, the method is coupled to an additional analytical method comprising gas chromatography, liquid chromatography, spectroscopy, microscopy, or a combination thereof.

In some aspects, disclosed herein is a method of identifying a biological polymer, the method comprising performing a de novo sequencing method comprising the steps of any preceding aspect.

In some aspects, disclosed herein is a computerized method of internally calibrating or correcting a raw mass-to-charge (m/z) spectrum, the method comprising:
obtaining (or providing) a data file or data object comprising a raw mass-to-charge (m/z) spectrum;
performing a peak-picking algorithm by the processor to extract one or more centroided peaks from the raw mass-to-charge (m/z) spectrum;
performing a transformation operation of a m/z value of the one or more centroided peaks into a charge-dependent value that enables invariant comparison between the one or more centroided peaks;
generating a charge pattern vector by the processor, wherein the one or more peaks are grouped to match a natural log-space spacing pattern across multiple charge states, and wherein one or more integer charge states are assigned to the one or more peaks based on their position within a matched pattern;
calculating expected positions of peaks based on a calibration model comprising a frequency-to-m/z conversion equation; and
adjusting one or more parameters of the calibration model to minimize deviations between observed and expected charge patterns, thereby internally calibrating the raw spectrum without the use of external calibrants.

In some embodiments, the method is used prior to sequencing of a biological polymer. In some embodiments, the raw m/z spectrum is generated from the biological polymer. In some embodiments, the biological polymer comprises more than one charge. In some embodiments, the biological polymer comprises a polypeptide (including but not limited to an antibody, a glycoprotein, a hormone, an enzyme, a contractile protein, a structural protein, a storage protein, or a fragment thereof), a polynucleotide (including but not limited to deoxyribonucleic acid (DNA), ribonucleic acid (RNA), a chemically modified analog, or a fragment thereof), or a fragment thereof.

In some embodiments, the raw m/z spectrum is generated from a tandem (MS/MS) mass spectrometer. In some embodiments, the method improves mass accuracy and improves sequence alignments generated after calibration. In some embodiments, the method minimizes deviations in ln-space charge spacing between corresponding isotopologues assigned to different charge states. In some embodiments, the method detects and removes a false peak that does not conform to predicted charge spacing or frequency-m/z relationships. In some embodiments, the method is used for drug testing, drug discovery, contaminant detection, clinical diagnostics, identification of pathological molecules, or a combination thereof.

In some embodiments, a calibration logic is implemented in a software configured to preprocess mass spectra from an ion-trapping instrument. In some embodiments, calibration is applied during acquisition in real time.

In some embodiments, present disclosure provides charge state difference calibration. It should be understood that the mass-invariant charge pattern in natural log-transformed m/z space provides a powerful means for internal calibration, particularly for intact proteins. Furthermore, the charge state difference calibration is valuable for ion trapping instruments with limited charge capacity such as, for example Fourier Transform-Ion Cyclotron Resonance (FT-ICR) or Orbitrap mass analyzers, wherein space charge effects can distort measured frequencies and lead to systematic m/z shifts. It should also be noted that by adjusting the observed peak positions to better match a known pattern, one of skill in the art can effectively mitigate space charge effects and perform accurate internal calibration without relying on known calibrants.

The present disclosure provides systems and non-transitory computer-readable medium (CRM) for performing de novo sequencing.

In some aspects, disclosed herein is a system comprising:
at least one processor; and
a memory operably coupled to least one processor, wherein the memory has computer executable instructions stored thereon that, when executed by least one processor, cause at least one processor to:
receive a data file comprising a raw mass-to-charge (m/z) spectrum
apply a peak-picking algorithm to extract one or more centroided peaks from the raw m/z spectrum;
perform a transformation operation of a mass-to-charge (m/z) value into a charge-dependent value that enables invariant comparison between the one or more centroided peaks;
generate a charge pattern vector, wherein the one or more peaks are grouped to match a natural log-space spacing pattern across multiple charge states, and wherein one or more integer charge states are assigned to the one or more peaks based on their position within a matched pattern;
output nearby fragment peak clusters relative to a control peak with a defined threshold;
and assemble fragment peak clusters from consecutive residue mass shifts to identify a biological polymer.

In some aspects, disclosed herein is a non-transitory computer-readable medium (CRM) having instructions stored thereon, wherein execution of the instructions by a processor causes the processor to:
receive a data file comprising a raw mass-to-charge (m/z) spectrum
apply a peak-picking algorithm to extract one or more centroided peaks from the raw m/z spectrum;
perform a transformation operation of a mass-to-charge (m/z) value into a charge-dependent value that enables invariant comparison between the one or more centroided peaks;
generate a charge pattern vector, wherein the one or more peaks are grouped to match a log-space spacing pattern across multiple charge states, and wherein one or more integer charge states are assigned to the one or more peaks based on their position within a matched pattern;
output nearby fragment peak clusters relative to a control peak with a defined threshold; and
assemble fragment peak clusters from consecutive residue mass shifts to identify a biological polymer.

In some embodiments, the system and/or the non-transitory CRM comprises a transformation operation that comprises a function in the form of Equation 1 or its equivalence:

$$ln ( \frac{m}{z} - q ) \qquad ( Equation 1 )$$

wherein q is a charge carrier mass.

In some embodiments, the method does not require monoisotopic mass assignment, isotopic deconvolution into a monoisotopic or average mass spectrum, or sequence database matching.

In its most basic configuration, the computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a programmable processor or a graphic processing unit that performs arithmetic and logic operations necessary for the operation of the computing device. While only one processing unit is shown, multiple processors (CPU, GPU, AI chip) may be present. As used herein, processing unit and processor refer to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

Computing devices may have additional features/functionality. For example, the computing device may include additional storage such as removable storage and non-removable storage, including, but not limited to, magnetic or optical disks or tapes. Computing devices may also contain network connection(s) that allow the device to communicate with other devices, such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing devices may also have input device(s) such as keyboards, keypads, switches, dials, mice, trackballs, touch screens, voice recognizers, card readers, paper tape readers, or other well-known input devices. Output device(s) such as printers, video monitors, liquid crystal displays (LCDs), touch screen displays, displays, speakers, etc., may also be included. The additional devices may be connected to the bus in order to facilitate the communication of data among the components of the computing device. All these devices are well-known in the art and need not be discussed at length here.

The computing device may be configured to execute program code encoded in tangible, computer-readable media. Tangible, computer-readable media refer to any media that is capable of providing data that causes the computing device (i.e., a machine) to operate in a particular fashion. Various computer-readable media may be utilized to provide instructions to the processing unit for execution. Example tangible, computer-readable media may include, but are not limited to, volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture to store and execute the software components presented herein. It should also be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

The exemplary system and method may be implemented (1) as a sequence of computer-implemented acts or program modules running on a computing system and/or (2) as interconnected machine logic circuits or circuit modules within the computing system. The logical operations described herein are referred to variously as state operations, acts, or modules. These operations, acts, and/or modules can be implemented in software, in firmware, in special-purpose digital logic, in hardware, and any combination thereof. It should also be appreciated that more or fewer operations can be performed than shown in the figures and described herein. These operations can also be performed in a different order than those described herein.

Although the system shown in the figures contains a local computing device, other embodiments may utilize a network interface to transmit the data to another computing device. As used herein, the term “network interface” refers to any signal, data, and/or software interface with a component, network, and/or process. By way of non-limiting example, a network interface may include one or more of FireWire (e.g., FW400, FW110, and/or other variation.), USB (e.g., USB2), Ethernet (e.g., 10/100, 10/100/1000 (Gigabit Ethernet), 10-Gig-E, and/or other Ethernet implementations), MoCA, Coaxsys (e.g., TVnet™), radio frequency tuner (e.g., in-band or OOB, cable modem, and/or other protocol), Wi-Fi (802.11), WiMAX (802.16), PAN (e.g., 802.15), cellular (e.g., 3G, LTE/LTE-A/TD-LTE, GSM, and/or other cellular technology), IrDA families, and/or other network interfaces. As used herein, the term “Wi-Fi” includes one or more of IEEE-Std. 802.11, variants of IEEE-Std. 802.11, standards related to IEEE-Std. 802.11 (e.g., 802.11 a/b/g/n/s/v), and/or other wireless standards. As used herein, the term “wireless” means any wireless signal, data, communication, and/or other wireless interface. By way of non-limiting example, a wireless interface may include one or more of Wi-Fi, Bluetooth, 3G (3GPP/3GPP2), HSDPA/HSUPA, TDMA, CDMA (e.g., IS-95A, WCDMA, and/or other wireless technology), FHSS, DSSS, GSM, PAN/802.15, WiMAX (802.16), 802.20, narrowband/FDMA, OFDM, PCS/DCS, LTE/LTE-A/TD-LTE, analog cellular, CDPD, satellite systems, millimeter wave or microwave systems, acoustic, infrared (i.e., IrDA), and/or other wireless interfaces.

Cloud System. The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Mass-Invariant Log-Transformed Mass Spectra

Most top-down proteomics workflows rely on deconvolution of intact and fragment ion m/z values using modeled isotope distributions, typically via an “averagine” approximation. This step often limits accuracy: poor fits to distorted isotope patterns can lead to incorrect monoisotopic mass assignment, widened mass tolerances, and inflated false discovery rates. To address these limitations, a framework/operation is employed for de novo sequencing and internal calibration that operates entirely in natural log-transformed m/z space-eliminating the need for monoisotopic mass determination.

By transforming spectra to ln(m/z−q), where q is the charge carrier mass, peaks arising from the same analyte mass align along a predictable pattern defined solely by charge state—a principle formalized by Jeong et al. in the FLASHDeconv algorithm. The mass-invariant spacing can be used to assign or re-assign charge states, pair isotopologues, and perform internal calibration without averagine-based fitting. Calibration is achieved by optimizing the A and/or B coefficient in the Ledford equation until observed peaks align with the expected −ln(c) spacing. Sequence tag inference is performed by comparing log-transformed peak positions from consecutive fragment ions to expected values based on known residue mass differences. When observed ln(m/z−q) values match those predicted for a given residue across multiple isotopologues and charge states, the corresponding mass difference can be confidently assigned—even from a single scan.

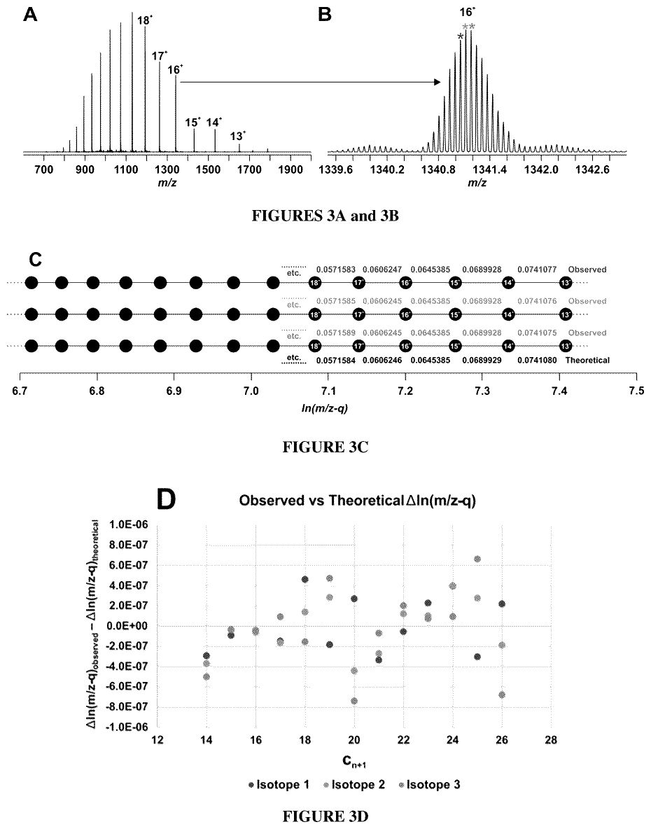

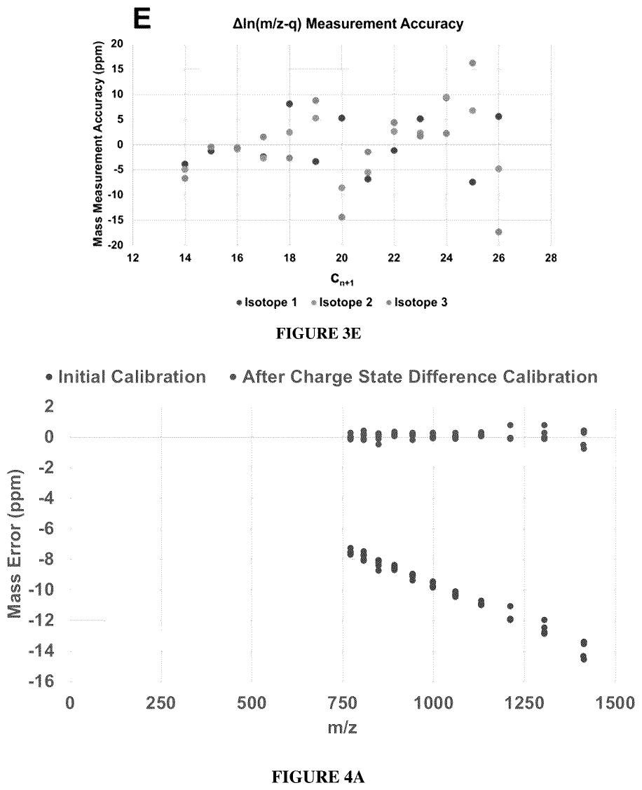

In an example implementation, the method was applied to 21 T FT-ICR MS/MS spectra of intact Protein G (FIG. 3), achieving agreement between predicted and observed values within approximately 1-20 ppm based on charge-state difference calibration (see FIG. 3E)

### Example 2: Mass-Invariant Log-Transformed Mass Spectra Enable De Novo Sequencing and Internal Calibration of Intact Proteins

Intact protein analysis presents persistent challenges due to the complexity of isotope distributions arising from the incorporation of heavy isotopes of carbon, hydrogen, nitrogen, oxygen, and sulfur. As protein mass increases, the probability of multiple heavy isotope incorporations rises, diminishing the relative abundance of the monoisotopic peak. In many cases—especially for low-abundance species or spectra with poor signal-to-noise—the monoisotopic peak may be weak or entirely undetectable. This complicates the process of determining accurate molecular mass, which is foundational to all top-down proteomic workflows.

A common workaround is to estimate the monoisotopic mass using the “averagine” model, which approximates an average elemental composition for amino acids to predict expected isotope distributions. While convenient, averagine-based fits can be shifted by one or more isotopologues if the monoisotopic peak is absent or misidentified, introducing systematic mass errors of 1-2 Da or more (FIG. 2). To accommodate these errors, database search tolerances must be widened, increasing the risk of false positives. Conversely, narrowing mass tolerances improves specificity but excludes true matches when mass estimates are inaccurate due to incorrect isotopic modeling. Additionally, mass measurement accuracy is inherently limited by the discrepancy between the true elemental composition of the analyte and the assumptions built into the averagine model. This issue is further compounded by electrospray ionization (ESI), which generates multiple charge states for each analyte, splitting signal intensity across overlapping mass-to-charge ratio (m/z) values, requiring high-resolution and spectral averaging for accurate deconvolution.

Current protein identification strategies—whether based on database search, spectral libraries, or de novo sequencing—depend heavily on the accuracy of these monoisotopic mass assignments. In database-driven approaches, spectra are m/z-to-mass deconvolved via averagine fits, and the resulting experimental monoisotopic masses are compared against in silico predictions from protein sequence databases. This inherently limits identification to sequences already present in the database. As a result, proteoforms containing unexpected sequence variants or post-translational modifications (PTMs) are not accurately identified or remain unidentified. For samples derived from non-model organisms or for applications requiring discovery of novel proteoforms—such as antibody sequencing—de novo approaches are the only viable option.

However, top-down de novo sequencing itself remains limited by the same assumptions: most methods still rely on averagine-based deconvolution to derive fragment and precursor monoisotopic masses prior to sequence inference. The inability to reliably determine monoisotopic mass without prior compositional knowledge hinders both identification and accurate interpretation of spectra.

The method (and system) presented here provides a framework/operation for database-independent, de novo sequencing of intact proteins that does not require monoisotopic mass assignment or m/z-to-mass deconvolution via averagine fitting. The approach is inspired in part by the FLASHDeconv algorithm, which identifies charge-state series in spectra by searching for mass-invariant patterns in natural log-transformed m/z space. While FLASHDeconv uses the principle for spectral decharging, the framework/operation described here uses the operation to construct mass-difference networks directly from isotopically resolved tandem mass spectrometry (MS/MS) data. These networks enable the identification of sequence-informative relationships between peaks without requiring an accurate precursor mass, opening new possibilities for accurate, untargeted proteoform sequencing.

Importantly, the mass-invariant charge pattern in natural log-transformed m/z space also provides a powerful means for internal calibration of mass spectra from electrospray-ionized intact proteins. This is especially valuable in ion trapping instruments with limited charge capacity—such as FT-ICR and Orbitrap mass analyzers—where space charge effects can distort measured frequencies and lead to systematic m/z shifts. By adjusting observed peak positions to better match the expected charge-state pattern, it is possible to mitigate space charge effects and perform accurate internal calibration without the need for known calibrants. Although previous approaches have proposed strategies for internal calibration based on mass difference analysis, they do not leverage the mass-invariant charge pattern in log-transformed m/z space as described here.

### Methods

Accurate charge determination is essential for the exemplary framework/operation. The FLASHDeconv algorithm developed by Jeong et al. is open-source, platform-independent software implemented in OpenMS. It included three sub-algorithms: spectral decharging, deisotoping (via averagine fitting), and feature finding. Only spectral decharging is relevant here.

Briefly, the measured m/z of an analyte may be given by:

$$\begin{matrix}\frac{m}{z} = \frac{{m}_{N} + c q}{c} = \frac{{m}_{N}}{c} + q & ∴ \frac{m}{z} - q = \frac{{m}_{N}}{c}\end{matrix} \qquad ( Equation 2 )$$

In Equation 2, m_{N} is the neutral analyte mass, c is the charge state, and q is the charge carrier mass (1.007276 Da).

Log-transforming m/z may yield:

$$ln ( \frac{m}{z} - q ) = ln ( \frac{{m}_{N}}{c} ) = ln ( {m}_{N} ) - ln ( c ) \qquad ( Equation 3 )$$

In Equation 3, for a given m_{N}, the distance between charge states in transformed space may form a universal charge pattern vector:

$$U := ( - ln ( {c}_{min} ) , - ln ( {c}_{min} + 1 ) , … , - ln ( {c}_{max} ) ) \qquad ( Equation 4 )$$

The approach/operation per Equation 4 can be employed to provide rapid identification of peaks corresponding to the same mass across multiple charge states.

Since the expected spacing between charge states is precisely known, differences between peaks arising from the same mass can be used to perform an internal calibration of the spectrum in natural log-transformed space. For Fourier-transform ion cyclotron resonance (FT-ICR) mass spectrometry, observed ion cyclotron frequency, f, may be converted to m/z via the Ledford equation, given below:

$$\frac{m}{z} = \frac{A}{f} + \frac{B}{{f}^{2}} \qquad ( Equation 5 )$$

In Equation 5, the A term is the magnetic field coefficient, and the B term is related to the electric trapping field and magnetron motion. By substituting this expression for m/z in Equation 2 and applying the natural log transformation, a calibration relationship can be derived between adjacent charge states (cₙ and cₙ₊₁) of the same mass:

$$ln ( {c}_{n + 1} ) - ln ( {c}_{n} ) = ln ( \frac{A}{{f}_{n}} + \frac{B}{{f}_{n}^{2}} - q ) - ln ( \frac{A}{{f}_{n + 1}} + \frac{B}{{f}_{n + 1}^{2}} - q ) \qquad ( Equation 6 )$$

Equation 6 can be employed to provide internal calibration based solely on the relative positions of isotopologue peaks across charge states, without requiring known calibrants or prior sequence knowledge.

Unlike methods that convert MS/MS data back to mass space, the framework/operation is based on an inference that amino acids can be sequenced directly from natural log-transformed m/z values. Following backbone fragmentation, fragment ions differ by the residue mass (RM) of individual amino acids. For fragments with the same charge state, the natural log-transformed values may follow:

$${ln ( \frac{m}{z} - q )}_{n \pm 1} = ln ( {( \frac{m}{z} - q )}_{n} \pm ( \frac{R M}{c} ) ) \qquad ( Equation 7 )$$

In Equation 7, this relationship may be used to provide direct detection of sequence-specific mass differences within the natural log-transformed space, eliminating the need for monoisotopic mass assignment or back-conversion to mass space.

In a study conducted to develop and evaluate the exemplary method, mass spectra were acquired with a custom-built 21 tesla (T) FT-ICR mass spectrometer at the National High Magnetic Field Laboratory (Tallahassee, FL). No phase correction was applied. Proteins were directly ionized by positive electrospray ionization via direct infusion and high-performance liquid chromatography.

### Results and Discussion

To evaluate the performance of this framework/operation, the study applied the operation to isotopically resolved top-down MS/MS spectra acquired from the recombinant standard proteins Apomyoglobin (equine, 17 kDa), Protein G (Streptococcus, 21 kDa), and Carbonic Anhydrase II (bovine, 29 kDa) using collision- and electron-based fragmentation. All spectra were analyzed without monoisotopic mass assignment, database search, or averagine fitting. Instead, peaks were mapped directly into ln(m/z−q) space. In this transformed space, both sequence-informative residue mass differences and charge-state alignment patterns were evaluated for consistency with theoretical expectations.

FIG. 3 demonstrates the mass-invariant charge pattern and its application for charge state assignment of intact Protein G. Pairwise comparisons of peak positions reveal natural log-domain differences that match known −ln(c) intervals to six decimal places. Unlike conventional deconvolution algorithms that fit isotope envelopes based on assumed elemental compositions, this method relies solely on geometric relationships between peaks in log space, making it broadly applicable to unknown analytes and discovery-mode proteomics workflows. This is particularly valuable in complex or low-S/N spectra and establishes a robust foundation for downstream calibration and sequence inference.

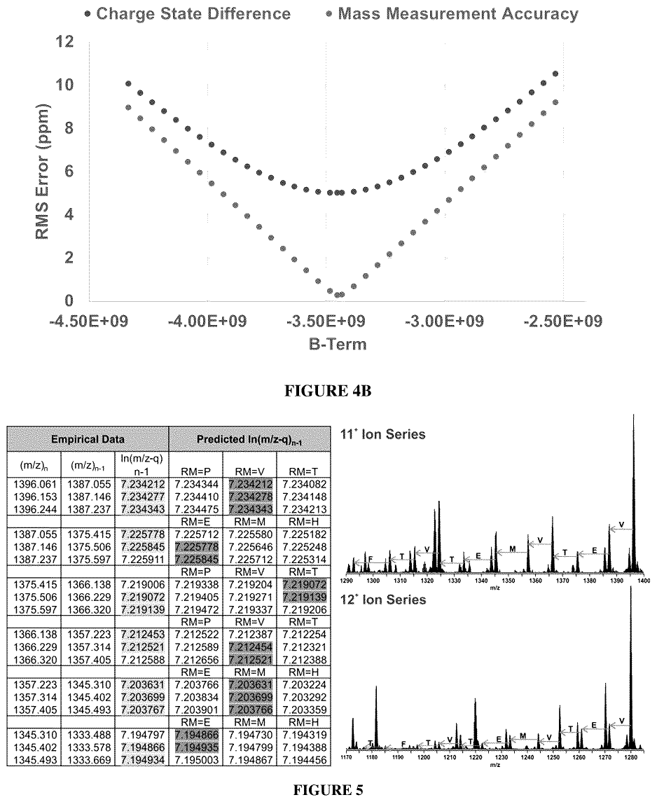

FIG. 4 demonstrates the use of the charge pattern vector to internally calibrate a distorted spectrum of Apomyoglobin. The initial A and B terms (A=323038592.7 Th*Hz; B=−2432564453.125 Th*Hz²) result in a systematic shift in mass measurement error with m/z indicative of a global frequency shift across all ions (FIG. 4A, red). The charge state difference determined from Equation 6 is minimized by iteration of the B term. FIG. 4B shows that the charge state difference and mass measurement error track together with a change in B-term and reach a minimum at nearly the same point. The mass measurement error with the new B term after charge state difference correction is shown in FIG. 4A (blue). The slope of the error has been flattened out, which indicates the global space charge-induced frequency shift has been corrected. The improvement in mass measurement error will be determined by how well the ion population is controlled and matched to the calibrated ion number.

A single MS/MS acquisition provides a stochastic snapshot of the ion population present in the mass analyzer at that moment. For highly charged proteins analyzed using ion-trapping instruments with limited charge capacity, isotopologue abundances can vary significantly between scans. In such cases, the relative intensities of isotope peaks cannot be relied upon to assign corresponding isotopologues between consecutive fragment ion peak clusters-particularly when the monoisotopic peak is weak or undetected. This presents a major obstacle for accurate mass-difference analysis in discovery-mode experiments, where analyte identity and composition are unknown. In contrast to averagine-based methods, which depend on statistically accurate isotope distributions, the approach presented here compares charge-resolved isotopologues within each peak cluster without relying on their relative abundances.

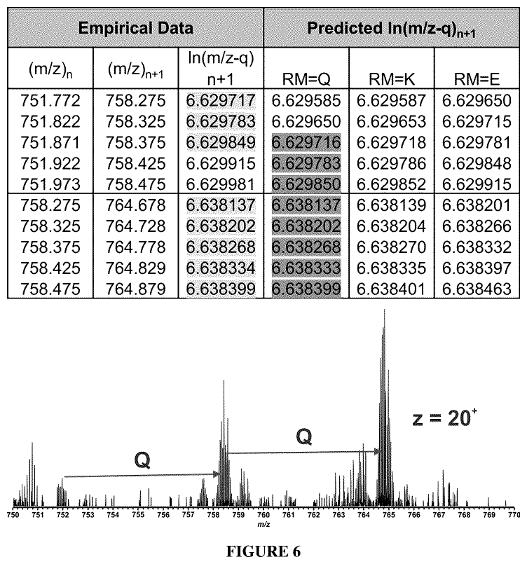

FIG. 5 illustrates the application of the charge-resolved, natural log-transformed mass-difference framework to a collision-induced dissociation MS/MS spectrum of Protein G. The two left-most columns of the table list the empirical m/z values of the three most abundant isotopologues for a series of seven consecutive fragment ions. Predicted ln(m/z−q)ₙ₋₁ were calculated from the observed (m/z)ₙ values using Equation 7. Observed ln(m/z−q)ₙ₋₁ were then compared to predicted values to assess isotopologue correspondence. In all cases, at least two of the three measured isotopologues matched the predicted value within ±1×10⁻⁶, confirming correct isotopologue pairing between consecutive fragment ion clusters. With this approach, a ten-residue sequence tag (VETVMETVTF (SEQ ID NO: 1)) was derived from the 11⁺ charge state. The same tag, extended by one residue (VETVMETVTFT (SEQ ID NO: 2)), was independently determined from the 12⁺ fragment ion series. The method also enabled resolution of near-isobaric residues—specifically glutamine (Q) and lysine (K)—in a single-scan MS/MS spectrum of Carbonic Anhydrase II (FIG. 6). These results demonstrate that Equation 7 enables direct identification of residue mass differences from raw log-transformed data, supporting accurate de novo sequencing from a single, minimally averaged spectrum—without the need for monoisotopic mass determination.

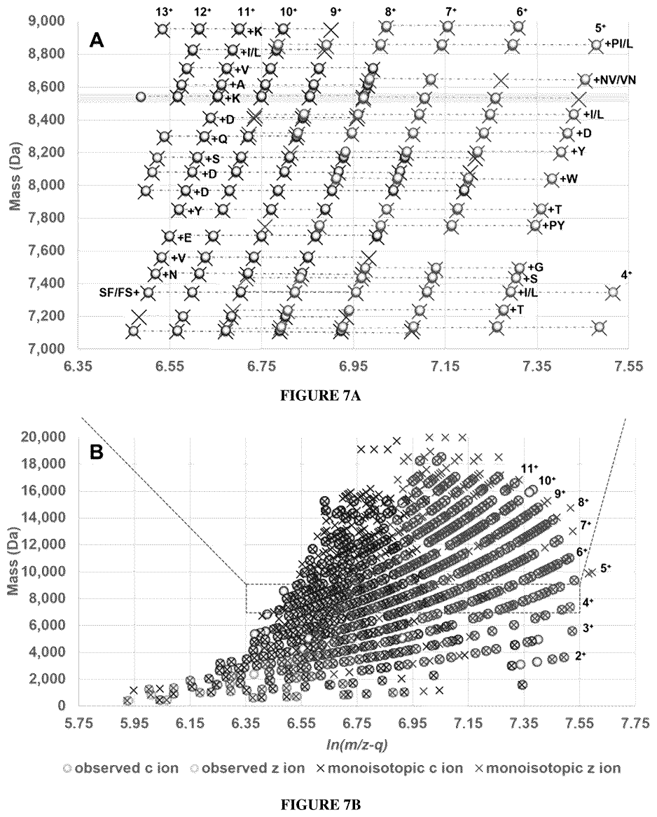

To visualize the organization of fragment ions in natural log-transformed space, c- and z•-type fragments from a 1500-scan, 6 ms ETD spectrum of Carbonic Anhydrase II (29 kDa) were plotted in ln(m/z−q) versus mass space (FIG. 7). The monoisotopic mass and ln(m/z−q) of manually identified fragments is represented with an “x”. A total of 492 c-ions and 548 z•-ions were identified. The spectrum was subsequently analyzed with ExDViewer (Beckman et al. 2021) for charge state assignment of fragment isotopologue peaks in m/z-space. The m/z and charge states of c- and z•-ion peaks matched to the sequence were used to calculate the masses of individual isotopologues with Equation 8:

$${m}_{N} = {z e}^{l n ( \frac{m}{z} - q )} \qquad ( Equation 8 )$$

The data are shown as circles. In the representation—referred to here as a natural log-transformed charge-mass plot—each fragment ion series forms a distinct curved path determined by its charge state. The observed isotopologue clusters align vertically above their corresponding theoretical monoisotopic positions (FIG. 8), with the curvature of each trajectory reflecting the mass-dependent relationship between ln(m/z−q) and fragment ion e sequence tags are deduced by examining mass differences between fragment peak clusters along each curve. For Carbonic Anhydrase, the distinct separation of fragment ion series in the 11⁺ and 5⁺ charge states can be attributed to differences in the rate that c- and z•-ions gain charge as mass increases (e.g., the number of Lys, Arg, and His residues grows faster in the “forward” N- to C-terminal direction). Sequence tag confidence increases when mass differences are confirmed by more than one isotopologue pair, or when the same residue series is detected in multiple charge states.

### Example 3: Step-by-Step Guide: Mass-Invariant, Log-Transformed Framework for Intact Protein Sequencing and Internal Calibration

The exemplary method may be used to provide direct identification of protein primary structure mass differences and internal calibration of isotopically resolved top-down FT-ICR MS and MS/MS spectra without requiring monoisotopic mass assignment, mass-domain deconvolution, or database matching. The study performed the following operation.
Step 1. Acquire a High-Resolution MS/MS Spectra. The step may include generating an MS/MS spectra (MS¹ and MS^{N}) of an intact protein or large fragment (e.g., antibody fragments) of a protein. The study ensured sufficient resolution to resolve individual isotopologues of multiply charged fragment ions.
Step 2. Perform Peak Picking with a Defined S/N or Abundance Threshold. The step may include applying a peak-picking algorithm to extract centroided peaks from the raw profile-mode spectrum. The study set a minimum intensity or signal-to-noise threshold to exclude noise peaks. Typical thresholds range from S/N≥3 to S/N≥10, depending on instrument and dynamic range. The study retained all peaks above the threshold for further analysis.
Step 3. Transform Peak m/z Values to ln(m/z−q) Space. For each peak, the step may include computing ln(m/z−q), where m/z is the observed mass-to-charge ratio, and q is the mass of the charge carrier (typically a proton, 1.007276 Da, in positive ion mode). This transformation linearizes charge-dependent peak spacing and enables mass-invariant comparison between peaks. The study stored the transformed values along with the original m/z, and intensity.
Step 4. Assign Charge States Using the Mass-Invariant Universal Charge Pattern. The step may include constructing a universal charge pattern vector U, defined per Equation 9. Equation 9 is an operational restatement of the universal charge pattern defined in Equation 4, expressed for an arbitrary ordered set of charge states (c₁, c₂, . . . , cₙ).

$$U := [ - ln ( {c}_{1} ) , - ln ( {c}_{2} ) , … , - ln ( {c}_{n} ) ] \qquad ( Equation 9 )$$

For each peak in the ln(m/z−q) spectrum, the step includes identifying potential charge-state series by comparing pairwise peak differences to elements of U. The study grouped the peaks that match the expected log-space spacing pattern across multiple charge states. The study assigned integer charge states to grouped peaks based on their position within the matched pattern and only retained peaks for which charge assignment was internally consistent with the log-space charge spacing.
Step 5. Perform Internal Calibration by Optimizing the Ledford B Coefficient in Log Space (Optional but Recommended). For spectra with no associated frequency data, for each peak, the step may include converting observed m/z to ion cyclotron frequency (f) using the inverse of the Ledford equation: m/z=A/f+B/f²→ (m/z)f²−Af−B=0 (quadratic equation), where A is the magnetic field coefficient and B accounts for the electric trapping field and magnetron motion. Store frequency values. The study adjusted the B coefficient iteratively to minimize the deviation between observed ln(m/z−q) values and the expected −ln(c) positions for peaks derived from the same analyte mass.

$$ln ( {c}_{n + 1} ) - ln ( {c}_{n} ) = ln ( \frac{A}{{f}_{n}} + \frac{B}{{f}_{n}^{2}} - q ) - ln ( \frac{A}{{f}_{n + 1}} + \frac{B}{{f}_{n + 1}^{2}} - q ) \qquad ( Equation 6 )$$

The study used the following equation (assume c₁−c₂=1):

$$( \frac{{c}_{2}}{{f}_{2}} - \frac{{c}_{1}}{{f}_{1}} ) A + ( \frac{{c}_{2}}{{f}_{2}^{2}} - \frac{{c}_{1}}{{f}_{1}^{2}} ) B = ( {c}_{2} - {c}_{1} ) q = - q \qquad ( Equation 10 )$$

The study contemplated that optimization can be performed by least-squares fitting of observed peak spacings to the U vector in natural log space. Once the best-fit B value is found, the study reapplied the Ledford equation and updated ln(m/z−q) coordinates. This aligns the observed peaks with the expected mass-invariant charge pattern. The study noted that no external calibrants or sequence knowledge were required.
Step 6. Predict Expected Positions of Adjacent Fragments in MS² Data. For each fragment peak at charge state c, the step may include computing the expected ln(m/z−q) value of adjacent fragments assuming a given residue mass (RM) or RM plus a chemical modification (e.g., phosphorylation, acetylation, methylation, etc.).

$${ln ( \frac{m}{z} - q )}_{n \pm 1} = ln ( {( \frac{m}{z} - q )}_{n} \pm ( \frac{R M}{c} ) ) \qquad ( Equation 7 )$$

Step 8. Match Predicted and Observed ln(m/z−q) Values. The step may include searching for ln(m/z−q) peaks in nearby fragment peak clusters that match predicted values within a tight tolerance (e.g., ±1×10⁻⁶). The study accepted a match when ≥2 isotopologue pairs support the same residue mass shift. The study discarded comparisons that do not meet this criterion.
Step 9. Assemble Sequence Tags from Consecutive Residue Mass Shifts. The step may include using matched residue masses between consecutive fragments to build a sequence tag. The study combined tags across charge states and isotopologues when possible. The study observed confidence increased with: agreement across multiple isotopologues, tag redundancy across charge states, and tag length (e.g., ≥5 residues).
Step 10. Identify Proteoform Families in Log Space Using MS¹ Data. The step may include grouping charge series for each precursor by identifying peaks that match the expected −ln(c) spacing, indicating they originate from the same analyte mass. Each group may correspond to one intact proteoform, and its position in natural log space is determined by its mass, e.g., per Equation 3 (reproduced below).

$$ln ( \frac{m}{z} - q ) = ln ( \frac{{m}_{N}}{c} ) = ln ( {m}_{N} ) - ln ( c ) \qquad ( Equation 3 )$$

The study compared the ln(m/z−q) positions of different charge series (i.e., different proteoforms) to identify consistent mass shifts in log space. These shifts may indicate reproducible mass differences between intact species, such as those introduced by post-translational modifications (PTMs), without the need to deconvolve to neutral mass. Proteoform families can thus be identified directly in ln(m/z−q) space based on shared charge-state patterns and consistent spacing offsets.

### Example 4: A Precise De Novo Sequencing Method for Top-Down Proteomics

Identification/sequencing/primary structure determination of peptides, proteins, oligonucleotides, and glycans from isotopically resolved tandem mass spectrometry (MS/MS) data.

Regulation of nearly every cellular process is directly linked to the primary structure of the proteins involved. Comprehensive knowledge of protein primary structure cannot be derived from the genome because translation of mRNA into protein does not dictate the chemical composition of the final protein product. The ultimate mass spectrometry (MS)-based proteomics platform would be capable of unequivocally distinguishing closely related protein sequences while concurrently characterizing any post-translational modifications (PTMs), which requires intact protein analysis (top-down proteomics, TDP).

Intact protein mass measurement is complicated by complex isotopic distributions that result from the incorporation of heavy isotopes of C, H, N, O, and S. This is further exacerbated by the use of electrospray ionization (ESI) for sample introduction to the mass analyzer. ESI ionizes proteins into multiple charge states. Therefore, analyte signals are split among several mass-to-charge ratio (m/z) values. As a result, spectra typically contain many overlapping signals that are difficult to resolve, deconvolve, and interpret, requiring high resolving power and extensive spectral averaging to improve signal-to-noise and isotope distribution fidelity.

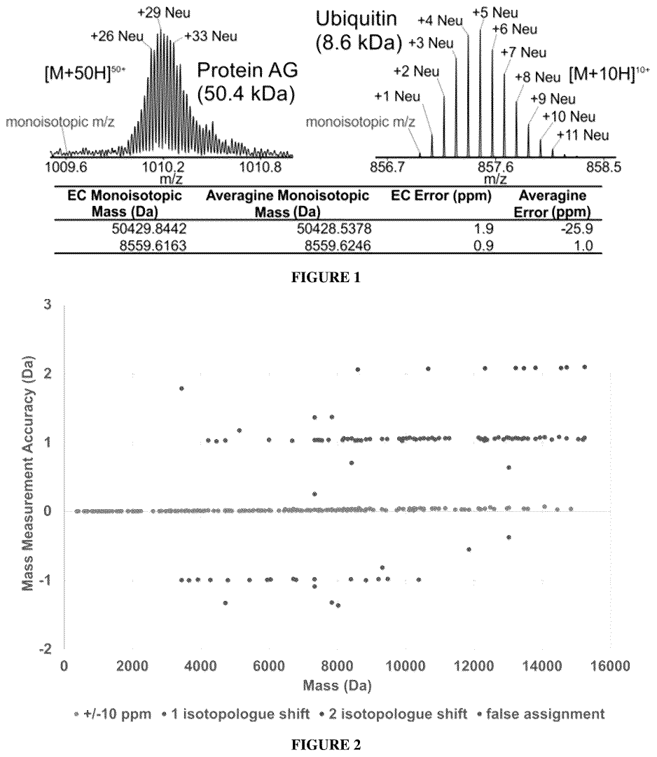

The masses of individual isotopologues and the width of an isotope distribution are governed by the elemental composition of the analyte. In the absence of this knowledge (i.e., “discovery” or “untargeted” proteomics), it is not possible to accurately determine an analyte's monoisotopic mass from experimental mass spectrometry data unless the monoisotopic peak of an isotope distribution can be identified as such and directly measured (FIG. 1). A common strategy for dealing with this is to use “averagine” distributions. Averagine is a virtual amino acid based on the statistical occurrences of amino acids in nature. Fit of the experimentally observed isotope distribution to an averagine distribution of similar nominal mass provides a reasonable estimate of protein monoisotopic mass. However, there are several limitations of this approach that are particularly detrimental to TDP analysis. When the monoisotopic peak is of low relative abundance or not observed, averagine fits can be shifted by 1-2 isotopologues (or 1-2 Da). As a result, mass tolerances must be widened to accommodate the error introduced by improper averagine fitting. This procedure is detrimental to proteoform identification, as the false discovery rate increases as mass tolerances are widened. Conversely, limiting mass tolerance to 10 ppm leaves a significant number of false negative assignments “on the table”. Mass measurement accuracy is also limited to the difference between the averagine and true elemental compositions.

The present disclosure provides a method for de novo sequencing peptides and proteins from isotopically resolved mass spectra without the need for m/z-to-mass deconvolution via averagine fitting. The invention was partially inspired by the FLASHDeconv algorithm. FLASHDeconv is implemented in C++ as a part of OpenMS and available as platform-independent open-source software under a three-clause license BSD at OpenMS.org/FLASHDeconv. It consists of three sub-algorithms: spectral decharging, deisotoping, and feature finding. Only the spectral decharging algorithm is relevant to this work. The algorithm identifies all peaks in a spectrum arising from the same protein mass (i.e., peaks only differ from one another by their charge state) by searching for a mass-invariant charge pattern in natural log-transformed m/z space.

The m/z of an analyte (empirically measured by the mass spectrometer) is given by Equation 2, where m is the mass of the analyte (e.g. a peptide/protein or fragment of a peptide/protein), c is the charge state (the integer number of charges the analyte possesses), and q is the mass of the charge carrier (a proton, 1.00727647 Da, in positive mode).

$$\frac{m}{z} = \frac{{m}_{N} + c q}{c} = \frac{{m}_{N}}{c} + q ∴ \frac{m}{z} - q = \frac{{m}_{N}}{c} \qquad ( Equation 2 )$$

In Equation 2, m_{N} is the neutral analyte mass, c is the charge state, and q is the charge carrier mass (1.007276 Da).

Log-transforming m/z may yield:

$$ln ( \frac{m}{z} - q ) = ln ( \frac{{m}_{N}}{c} ) = ln ( {m}_{N} ) - ln ( c ) \qquad ( Equation 3 )$$

In Equation 3, for a given m_{N}, the distance between any peaks in the transformed space may be defined by a universal charge pattern vector, U:

$$U := ( - ln ( {c}_{min} ) , - ln ( {c}_{min} + 1 ) , … , - ln ( {c}_{max} ) ) \qquad ( Equation 4 )$$

The pattern may be used to quickly detect peaks from the same mass with distinct charge states. The FLASHDeconv algorithm similarly removes harmonic artifacts from the spectrum, and then proceeds to the next sub-algorithm, where the spectrum is transformed into the mass space for centroiding and monoisotopic mass determination via averagine fitting.

This disclosure describes a novel method for de novo sequencing intact proteins by MS/MS. Instead of returning to the mass space, the amino acid sequence is determined directly from the natural log-transformed MS/MS spectrum. The method assumes that the charge states of isotopic peak clusters have been accurately determined via either universal charge pattern vector scanning, or by determining the mass differences between peaks in the same peak cluster.

To sequence a protein by MS/MS, the protein is fragmented along its amide backbone to produce a collection of fragment ions that are measured to produce an MS/MS spectrum. Peaks corresponding to consecutive fragments of an ion series will differ by the residue mass (RM) of an amino acid. For the natural log-transformed spectrum, the ln(m/z−q) value for consecutive fragments is given by Equation 7.

$${ln ( \frac{m}{z} - q )}_{n \pm 1} = ln ( {( \frac{m}{z} - q )}_{n} \pm ( \frac{R M}{c} ) ) \qquad ( Equation 7 )$$

A comparison may be performed of the empirical ln(m/z−q) values to predicted values generated by iterating the RM. The values for all corresponding isotopologues of a peak cluster were observed to match the predicted values with high accuracy (FIG. 5 and Table 1).

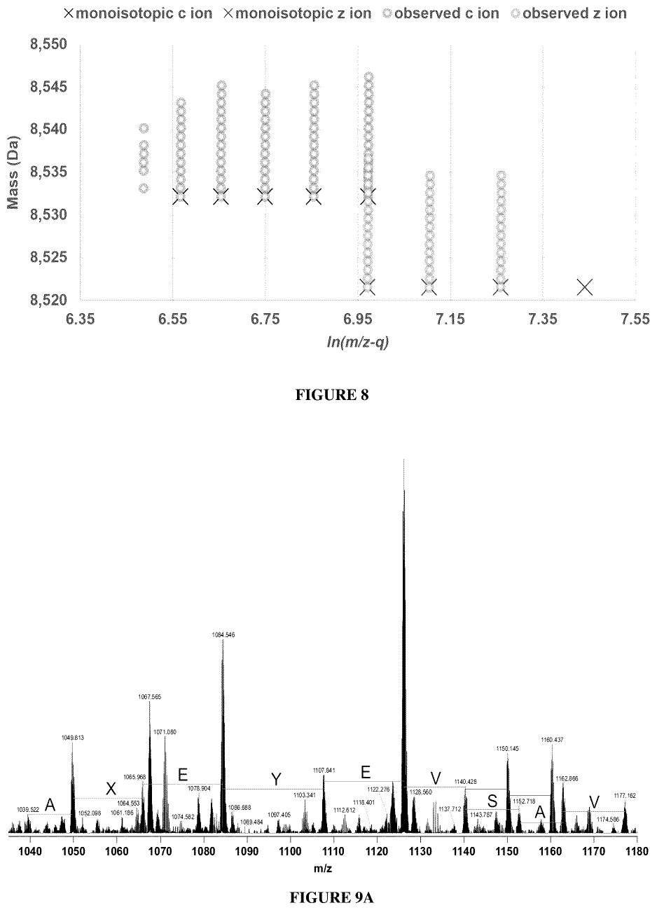

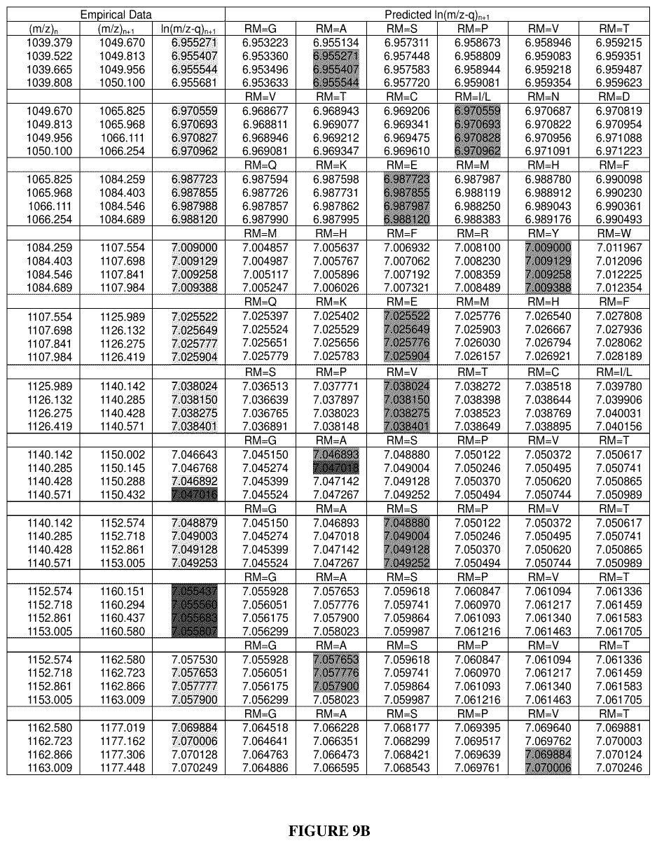

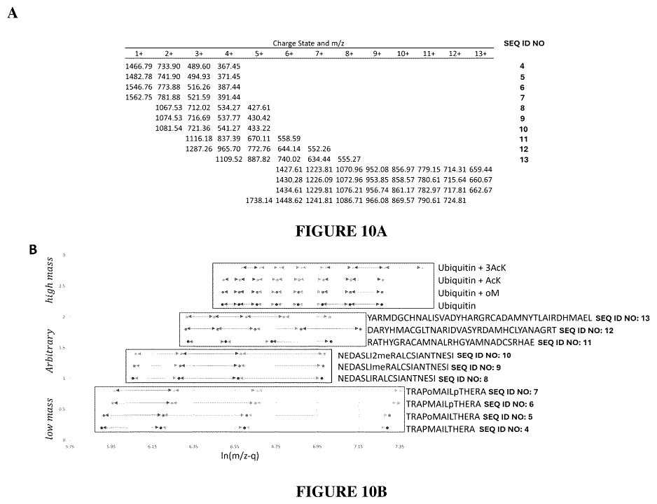

To continue the sequence analysis, candidate signals of the same charge state may be sequentially compared to predicted values, e.g., as shown in FIG. 5 and FIG. 9.

A single spectral acquisition is a stochastic measurement of the ions present inside the mass analyzer. An accurate statistical representation of isotopologue abundance cannot be achieved without spectral averaging, which requires precious instrument time. As a result, if the identity of the analyte is not known, there is no way to know which isotopologues of consecutive peak clusters correspond to one another (i.e., have the same number of heavy isotopes incorporated). This, in turn, makes it impossible to determine the mass differences between fragment ions directly from the spectrum with confidence. This method eliminates this requirement by considering the three most abundant isotopologues of a peak cluster simultaneously. Since observed values match predicted values to 1E-6, it is easy to determine which isotopologue peaks should be compared to one another. Confidence scoring metrics can be improved by considering additional isotopologues as required, and further synergies can be realized when sequence tags are observed in more than one charge state.

The de novo protein sequencing results can be integrated with a database search in which the sequence tag is aligned with protein database entries to determine the identity of the protein. Once the identity of the protein is known, additional information regarding the rest of the sequence, PTMs, mutations, isoforms, etc., can be retrieved and used to refine the performance of the de novo sequencing algorithm. For example, when the sequence tag is searched “forward” (VETVMETVTF (SEQ ID NO: 1)) against the SwissProt sequence database (contains 571,864 entries) without specifying the organism, no hits are returned with 100% query coverage. When the tag is searched “backward” (FTVTEMVTEV (SEQ ID NO: 3)), the best scoring match includes 100% of the queried sequence and is the correct protein, Immunoglobulin G-binding Protein G (Uniprot P06654) from the bacterium, Streptococcus. Since the sequence of the protein is now known, the fragment ion types and indices can be easily assigned (Table 1, b₁₄₀¹¹⁺, b₁₄₀¹²⁺, b₁₄₀¹³⁺; Table 2, b₁₄₀¹¹⁺-b₁₃₀¹¹⁺), and additional peaks can be quickly matched to the sequence.

The method can be adapted for the analysis of isotopically resolved MS/MS spectra derived from any biopolymer (proteins, oligonucleotides, polysaccharides) comprised of predictable monomers (amino acids, monosaccharides, nucleotides) and other chemical modifications so long as it is observed in multiple charge states. Popular peptide and protein identification approaches involve either database search, spectral libraries (for data-independent acquisition), or de novo sequencing. Database searches typically require two pieces of information-accurate intact and fragment ion masses. The experimental mass values are compared with theoretical (monoisotopic mass) values predicted in silico from protein sequences in a database and matched (or not matched) to an entry in the database within a specified mass tolerance. A spectral library search is similar, except a database of previously collected spectra is used instead of a sequence database. In both cases, analytes containing an unexpected sequence or PTM(s) cannot be identified correctly because they are not present in the databases employed. De novo sequencing, which involves direct interpretation of mass spectral data, is the only option for several important applications, including the study of organisms with unknown genomes, the discovery of novel splice variants, mutations, and post-translationally modified proteins, and sequencing complementarity-determining regions of antibodies.

Currently, database search is considered substantially more reliable for the identification of proteins by TDP. While a handful of de novo sequencing algorithms have been developed for TDP (Horn et al. 2000; Liu et al. 2014; Vyatkina et al. 2016; and He et al. 2018), none are commonly utilized, and all rely on deconvolution to deisotope spectra prior to interpretation. This method avoids errors introduced by misassignment of the monoisotopic mass following averagine m/z-to-m deconvolution at the intact and fragment ion levels, which should, in turn, lower false assignments. Errors introduced by comparison of non-corresponding isotopologues are also avoided. De novo sequencing methods benefit from the fact that no intact mass measurement is required. A sequence tag of just seven amino acids is typically sufficient to identify a protein. Finally, once implemented, this method will be computationally faster than a database or spectral library search.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLES
Table 1. The three most abundant isotopologues of series of 11 consecutive fragment ions of charge
state +11 are obtained from empirical data. The predicted ln(m/z − q)ₙ₋₁ values are calculated
with Equation 7 starting from the observed (m/z)ₙ values. The observed ln(m/z − q)ₙ₋₁ values
(italicized) are matched the predicted values (asterisk(*)). In some cases, only two of the three
values match because only two of the three represented isotopologues correspond to one another (bold).
A ten-residue sequence tag, VETVMETVTF (SEQ ID NO: 1), is derived from the data. The same sequence
tag can be derived from the +12 series of fragment ions in the same spectrum (data not shown).

| Empirical Data | | | Predicted In(m/z − q)ₙ₋₁ | | | | | |
|---|---|---|---|---|---|---|---|---|
| | | ln(m/z − | | | | | | |
| (m/z)ₙ | (m/z)ₙ₋₁ | q) n − 1 | RM = G | RM = A | RM = S | RM = P | RM = V | RM = T |
| 1396.061 | 1387.055 | 7.234212 | 7.236966 | 7.236048 | 7.235001 | 7.234344 | 7.234212* | 7.234082 |
| 1396.153 | 1387.146 | 7.234277 | 7.237031 | 7.236114 | 7.235067 | 7.234410 | 7.234278* | 7.234148 |
| 1396.244 | 1387.237 | 7.234343 | 7.237097 | 7.236180 | 7.235132 | 7.234475 | 7.234343* | 7.234213 |
| | | | RM = D | RM = Q | RM = K | RM = E | RM = M | RM = H |
| 1387.055 | 1375.415 | 7.225778 | 7.226639 | 7.225777 | 7.225775 | 7.225712 | 7.225580 | 7.225182 |
| 1387.146 | 1375.506 | 7.225845 | 7.226705 | 7.225843 | 7.225841 | 7.225778* | 7.225646 | 7.225248 |
| 1387.237 | 1375.597 | 7.225911 | 7.226771 | 7.225910 | 7.225907 | 7.225845* | 7.225712 | 7.225314 |
| | | | RM = G | RM = A | RM = S | RM = P | RM = V | RM = T |
| 1375.415 | 1366.138 | 7.219006 | 7.221999 | 7.221068 | 7.220005 | 7.219338 | 7.219204 | 7.219072* |
| 1375.506 | 1366.229 | 7.219072 | 7.222066 | 7.221135 | 7.220072 | 7.219405 | 7.219271 | 7.219139* |
| 1375.597 | 1366.320 | 7.219139 | 7.222133 | 7.221202 | 7.220138 | 7.219472 | 7.219337 | 7.219206 |
| | | | RM = G | RM = A | RM = S | RM = P | RM = V | RM = T |
| 1366.138 | 1357.223 | 7.212453 | 7.215201 | 7.214264 | 7.213193 | 7.212522 | 7.212387 | 7.212254 |
| 1366.229 | 1357.314 | 7.212521 | 7.215268 | 7.214331 | 7.213260 | 7.212589 | 7.212454* | 7.212321 |
| 1366.320 | 1357.405 | 7.212588 | 7.215335 | 7.214398 | 7.213327 | 7.212656 | 7.212521* | 7.212388 |
| | | | RM = D | RM = Q | RM = K | RM = E | RM = M | RM = H |
| 1357.223 | 1345.310 | 7.203631 | 7.204713 | 7.203832 | 7.203830 | 7.203766 | 7.203631* | 7.203224 |
| 1357.314 | 1345.402 | 7.203699 | 7.204781 | 7.203900 | 7.203898 | 7.203834 | 7.203699* | 7.203292 |
| 1357.405 | 1345.493 | 7.203767 | 7.204849 | 7.203968 | 7.203966 | 7.203901 | 7.203766* | 7.203359 |
| | | | RM = D | RM = Q | RM = K | RM = E | RM = M | RM = H |
| 1345.310 | 1333.488 | 7.194797 | 7.195822 | 7.194933 | 7.194931 | 7.194866* | 7.194730 | 7.194319 |
| 1345.402 | 1333.578 | 7.194866 | 7.195890 | 7.195002 | 7.194999 | 7.194935* | 7.194799 | 7.194388 |
| 1345.493 | 1333.669 | 7.194934 | 7.195958 | 7.195070 | 7.195068 | 7.195003 | 7.194867 | 7.194456 |
| | | | RM = G | RM = A | RM = S | RM = P | RM = V | RM = T |
| 1333.488 | 1324.301 | 7.190900 | 7.189939 | 7.188842 | 7.188154 | 7.188016 | 7.187880 | 7.190900* |
| 1333.578 | 1324.392 | 7.190968 | 7.190008 | 7.188911 | 7.188223 | 7.188084 | 7.187948 | 7.190968* |
| 1333.669 | 1324.484 | 7.191036 | 7.190076 | 7.188979 | 7.188291 | 7.188153 | 7.188017 | 7.191036* |
| | | | RM = G | RM = A | RM = S | RM = P | RM = V | RM = T |
| 1324.301 | 1315.295 | 7.181050 | 7.183954 | 7.182987 | 7.181882 | 7.181189 | 7.181050* | 7.180913 |
| 1324.392 | 1315.387 | 7.181120 | 7.184023 | 7.183056 | 7.181952 | 7.181259 | 7.181120* | 7.180983 |
| 1324.484 | 1315.478 | 7.181189 | 7.184093 | 7.183126 | 7.182021 | 7.181328 | 7.181189* | 7.181052 |
| | | | RM = G | RM = A | RM = S | RM = P | RM = V | RM = T |
| 1315.295 | 1306.109 | 7.174036 | 7.177098 | 7.176125 | 7.175012 | 7.174315 | 7.174174 | 7.174036* |
| 1315.387 | 1306.201 | 7.174107 | 7.177168 | 7.176194 | 7.175082 | 7.174385 | 7.174244 | 7.174106* |
| 1315.478 | 1306.292 | 7.174176 | 7.177238 | 7.176264 | 7.175152 | 7.174455 | 7.174314 | 7.174176* |
| | | | RM = M | RM = H | RM = F | RM = R | RM = Y | RM = W |
| 1306.109 | 1292.739 | 7.163739 | 7.164867 | 7.164443 | 7.163739* | 7.163103 | 7.162613 | 7.160990 |
| 1306.201 | 1292.830 | 7.163809 | 7.164938 | 7.164514 | 7.163810* | 7.163174 | 7.162684 | 7.161061 |
| 1306.292 | 1292.922 | 7.163880 | 7.165008 | 7.165388 | 7.163881* | 7.163245 | 7.162754 | 7.161132 |

SEQUENCES

1. SEQ ID NO: 1-Example Sequence Tag (Forward)
VETVMETVTF
2. SEQ ID NO: 2-Example Sequence Tag (Extended by one residue)
VETVMETVTFT
3. SEQ ID NO: 3-Example Sequence Tag (Reverse/Backward)
FTVTEMVTEV
4. SEQ ID NO: 4-Example Sequence #1
TRAPMAILTHERA
5. SEQ ID NO: 5-Example Sequence #2
TRAPoMAILTHERA “o” refers to oxidation of methionine (M)
6. SEQ ID NO: 6-Example Sequence #3
TRAPMAILpTHERA “p” refers to a phosphoryl group
7. SEQ ID NO: 7-Example Sequence #4
TRAPoMAILpTHERA “o” refers to oxidation of methionine (M); “p” refers
to phosphoryl group
8. SEQ ID NO: 8-Example Sequence #5
NEDASLIRALCSIANTNESI
9. SEQ ID NO: 9-Example Sequence #6
NEDASLImeRALCSIANTNESI “me” refers to a methyl group
10. SEQ ID NO: 10-Example Sequence #7
NEDASLI2meRALCSIANTNESI “2me” refers to 2 methyl groups
11. SEQ ID NO: 11-Example Sequence #8
RATHYGRACAMNALRHGYAMNADCSRHAE
12. SEQ ID NO: 12-Example Sequence #9
DARYHMACGLTNARIDVASYRDAMHCLYANAGRT
13. SEQ ID NO: 13-Example Sequence #10
YARMDGCHNALISVADYHARGRCADAMNYTLAIRDHMAEL
14. SEQ ID NO: 14-Nine residue sequence tag, where X refers to Leucine
(Leu)/Isoleucine (Iso) isomers
AXEYEVSAV

What is claimed is:

1. A computerized method of de novo sequencing of a biological polymer from a mass spectrometer, the method comprising:
providing a data file or data object comprising a spectra from a mass spectrometer;
performing a peak-picking algorithm by the processor to extract one or more centroided peaks from the spectra from the mass spectrometer;
performing a transformation operation of a m/z value of the one or more centroided peaks into a charge-dependent value that enables invariant comparison between the one or more centroided peaks;
generating a charge pattern vector by the processor, wherein the one or more peaks are grouped to match a spacing pattern across multiple charge states, and wherein one or more integer charge states are assigned to the one or more peaks based on their position within a matched pattern;
generating from the processor nearby fragment peak clusters relative to a control peak with a defined threshold; and
assembling fragment peak clusters, via the processor, from consecutive residue mass shifts to identify a full or partial sequence of a biological polymer by a de novo sequencing operation.

2. The method of claim 1, wherein the transformation operation comprises applying a natural logarithmic function in a form of:
$$ln ( \frac{m}{z} - q )$$
wherein q is a charge carrier mass.

3. The method of claim 1, wherein the biological polymer comprises a polypeptide, a polynucleotide, or a fragment thereof.

4. The method of claim 3, wherein the polypeptide comprises an antibody, a glycoprotein, a hormone, an enzyme, a contractile protein, a structural protein, a storage protein, or a fragment thereof.

5. The method of claim 3, wherein the polynucleotide comprises deoxyribonucleic acid (DNA), ribonucleic acid (RNA), a chemically modified analog, or a fragment thereof.

6. The method of claim 1, further comprising identifying one or more post-translational modifications of the biological polymer or the unknown biological polymer.

7. The method of claim 1, further comprising identifying one or more amino acid or nucleotide substitutions.

8. The method of claim 1, further comprising identifying one or more isoforms of the biological polymer.

9. The method of claim 2, wherein the charge carrier mass comprises an electron, a proton, a monoatomic ion, or a polyatomic ion.

10. The method of claim 1, wherein the spectra from the mass spectrometer is generated from a tandem (MS/MS) mass spectrometer.

11. The method of claim 1, wherein the method directly identifies an amino acid sequence tag directly from charge-resolved isotopologue peaks, without requiring monoisotopic mass assignment, collapsing isotopologue clusters into a deconvolved mass spectrum, or database matching.

12. The method of claim 1, wherein the method directly identifies a polynucleotide sequence tag directly from charge-resolved isotopologue peaks, without requiring monoisotopic mass assignment, collapsing isotopologue clusters into a deconvolved mass spectrum, or database matching.

13. The method of claim 1, wherein the method comprises internally calibrating the spectrum by minimizing charge state difference errors between corresponding isotopologues assigned to different charge states.

14. The method of claim 1, wherein the method identifies and removes a false peak that does not conform to predicted charge state or mass difference patterns from the spectra from the mass spectrometer.

15. The method of claim 1, wherein the method is used for drug testing, drug discovery, contaminant detection, clinical diagnostics, identification of pathological molecules, biomarkers, or a combination thereof.

16. A computerized method of internally calibrating or correcting a mass-to-charge (m/z) spectrum, the method comprising:
providing a data file or data object comprising a spectra from a mass spectrometer;
performing a peak-picking algorithm by the processor to extract one or more centroided peaks from the spectra from the mass spectrometer;
performing a transformation operation of a m/z value of the one or more centroided peaks into a charge-dependent value that enables invariant comparison between the one or more centroided peaks;
generating a charge pattern vector by the processor, wherein the one or more peaks are grouped to match a log-space spacing pattern across multiple charge states, and wherein one or more integer charge states are assigned to the one or more peaks based on their position within a matched pattern;
calculating expected positions of peaks based on a calibration model comprising a frequency-to-m/z conversion equation; and
adjusting one or more parameters of the calibration model to minimize deviations between observed and expected charge patterns, thereby internally calibrating the spectra from the mass spectrometer without the use of external calibrants.

17. The method of claim 16, wherein the biological polymer comprises a polypeptide, a polynucleotide, or a fragment thereof.

18. The method of claim 16, wherein the spectra from a mass spectrometer is generated from a tandem (MS/MS) mass spectrometer.

19. A system comprising:
at least one processor; and
a memory operably coupled to the at least one processor, wherein the memory has computer executable instructions stored thereon that, when executed by the at least one processor, cause at least one processor to:
provide a data file or data object comprising a raw mass-to-charge (m/z) spectrum
apply a peak-picking algorithm to extract one or more centroided peaks from the raw m/z spectrum;
perform a transformation operation of a mass-to-charge (m/z) value into a charge-dependent value that enables invariant comparison between the one or more centroided peaks;
generate a charge pattern vector, wherein the one or more peaks are grouped to match a log-space spacing pattern across multiple charge states, and wherein one or more integer charge states are assigned to the one or more peaks based on their position within a matched pattern;
output nearby fragment peak clusters relative to a control peak with a defined threshold; and
assemble fragment peak clusters from consecutive residue mass shifts to identify a biological polymer.

20. A non-transitory computer-readable medium (CRM) having instructions stored thereon, wherein execution of the instructions by a processor causes the processor to perform the method of claim 1.
